(19) United States
(12) Patent Application Publication
(10) Pub. No.: US 2004/0229816 A1
(43) Pub. Date: Nov. 18, 2004

(54) Anti-angiogenic and anti-tumoral properties of beta and gamma secretase inhibitors

(76) Inventors: Daniel Paris, Bradenton, FL (US); Michael Mullan, Bradenton, FL (US)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/780,905

(22) Filed: Feb. 18, 2004

Related U.S. Application Data
(60) Provisional application No. 60/319,954, filed on Feb. 18, 2003.

(51) Int. Cl. A61K 38/05

(52) U.S. Cl. 514/18

(57) ABSTRACT

The present invention relates to methods of treating tumors or proliferative disorders that are associated with angiogenesis by administering γ-secretase and β-secretase inhibitors that inhibit secretases involved in amyloid precursor protein processing. In particular, methods are provided to treat tumors or proliferative disorders, or to inhibit angiogenesis associated with tumors, proliferative or inflammatory disorders, in animals or humans in need of such treatment or angiogenic inhibition, by administering to the animal or human therapeutically effective amounts in unit dosage form of a composition containing a carrier and at least one γ-secretase or β-secretase inhibitor that inhibits secretase APP processing.

The present application claims priority to U.S. Provisional Application No. 60/319,954, filed Feb. 18, 2003, which is incorporated herein by reference.

### BACKGROUND OF THE INVENTION

1. Field of the Invention

This invention relates generally to methods for treating angiogenic-related disorders. More specifically, this invention relates to methods of treating tumors or proliferative disorders associated with angiogenesis, by administering compositions that inhibit such angiogenesis.

2. Description of Related Art

Angiogenesis, the formation of new capillaries from pre-existing blood vessels, is a fundamental process needed for normal growth, primarily in embryo development, during wound healing and in response to ovulation.

Angiogenesis is stimulated when hypoxic, diseased, or injured tissues produce and release angiogenic promoters such as vascular endothelial growth factor (VEGF), fibroblast growth factor (FGF)-1, angiogenin, epidermal growth factor (EGF), placental growth factor (PGF), platelet-derived growth factor (PDGF), and tumor necrosis factor alpha (TNF-alpha). This signaling activates certain genes in the host tissue that, in turn, make proteins that stimulate the growth of new blood vessels. Specifically, endothelial cells from preexisting vessels are “activated” by these proteins to release proteases, which results in the degradation of basement membrane surrounding the existing blood vessels. The endothelial cells then migrate into the interstitial space where they proliferate and differentiate into mature blood vessels (Carmeliet, P., Nature Med., 6, 389-395, 2000).

Normal regulation of angiogenesis is governed by a fine balance between factors that induce the formation of blood vessels and those that halt or inhibit the process. When this balance is destroyed, it usually results in pathological angiogenesis (the abnormal rapid proliferation of blood vessels), which causes increased blood vessel formation in diseases that depend on angiogenesis.

Pathological angiogenesis is a hallmark of cancer, but also occurs in various ischemic and inflammatory diseases, such as rheumatoid arthritis, psoriasis and asthma. Indeed, pathological angiogenesis is implicated in over twenty diseases. Additionally, obesity may be accompanied by angiogenesis because of the cellular hypoxia that usually accompanies the obese state. Obesity-related angiogenesis is believed to facilitate the deposition of fat.

Cancer represents the most extreme, life-threatening disease process in which blood vessel growth plays an important role. Without the formation of new blood vessels, solid tumors will not grow beyond a few millimeters, but with an enriched environment provided by the new blood vessels, tumors thrive. It is believed that there is a direct correlation between the density of tumor vessels and an adverse prognosis in patients with certain solid tumors, including breast, colon, lung, kidney, bladder and head and neck tumors.

Tumor angiogenesis, therefore, is the proliferation of a network of blood vessels that penetrates into cancerous growths, supplying nutrients and oxygen and removing waste products. Solid tumors smaller than 1 to 2 cubic millimeters are not vascularized and persist in situ for a long period of time (from months to years) in an avascular, quiescent status. In this phase the tumor may contain a few million cells. For cancer to metastasize, the tumor needs to be supplied by blood vessels that bring oxygen and nutrients and remove metabolic wastes. Beyond the critical volume of 2 cubic millimeters, oxygen and nutrients have difficulty diffusing to the cells in the center of the tumor, causing a state of cellular hypoxia that marks the onset of tumor angiogenesis.

It is known that some tumors secrete substances that can inhibit angiogenesis, known as angiogenic inhibitors. For example, some large tumors give off angiogenesis inhibitors that prevent blood vessels from sending branches to smaller, more distant tumors. Because angiogenesis is of crucial importance for tumor growth, inhibiting this process has become a major challenge in the development of new anti-cancer agents. Although the blood vessels of tumors have been studied since the early 1800s, it is only in the past few years that this approach as an effective therapy has become realistic. The concept of cancer anti-angiogenic therapy stems from the work of Dr. Judah Folkman in the early 1970s, a pediatric surgeon at the Children's Hospital in Boston. Dr. Folkman was the first to emphasize that solid tumors cannot grow beyond the size of a pinhead (1 to 2 cubic millimeters) without inducing the formation of new blood vessels to supply the nutritional and other needs of the tumor. He recognized that inhibiting the growth of tumor blood vessels could lead to effective methods in attacking malignancy. This theory, now confirmed by a large body of experimental evidence, implies that tumors can potentially be starved to death by inhibiting their blood supply.

Thus, there is the potential to treat a wide variety of cancers, as well as other angiogenic-related disorders, by inhibiting the process of angiogenesis in the patient.

Theoretically, such treatment should only affect the pathological formation of new blood vessels associated with angiogenic-related diseases, because once a person has stopped growing, their blood vessel system is basically stable and only grows to repair an injury. Some of the naturally occurring inhibitors of angiogenesis include angiostatin, endostatin, interferons, platelet factor 4, thrombospondin, transforming growth factor beta, and tissue inhibitor of metalloproteinase. The potential important benefits of using angiogenic inhibitors for cancer treatment over standard chemotherapy are the lack of resistance to therapy and the lack of significant side effects on other normal tissues. These angiogenic inhibitors, however, may not necessarily kill tumors, but rather hold them in check indefinitely, making it necessary to continue therapy with angiogenic inhibitors for the life of the individual or use in combination with other standard chemotherapy drugs.

Amyloid precursor protein (APP) is a large glycoprotein that is highly expressed in neurons but also is found in vascular cells, including endothelial cells. Indeed, APP is expressed very early during fetal life in the endothelia of neovascularized tissue, and particularly in cerebral endothelia (Ott, M. O. et al., Genes Evol., 211, 355-7, 2001), which suggests a normal role for APP and/or its metabolites in early angiogenesis. APP is a single-transmembrane protein with a 590-680 amino acid extracellular amino terminal domain and an approximately 55 amino acid cytoplasmic tail. APP mRNA from the APP gene on chromosome 21 undergoes alternative splicing to yield eight possible isoforms, three of which (the 695, 751 and 770 amino acid isoforms) predominate in the brain. APP undergoes proteolytic processing via three enzymatic activities, termed α-, β- and γ-secretase. Alpha-secretase cleaves APP at amino acid 17 of the amyloid-beta (Aβ) domain, thus releasing the large soluble amino-terminal fragment α-APP for secretion. Because α-secretase cleaves within the Aβ domain, this cleavage precludes Aβ formation. Alternatively, APP can be cleaved by β-secretase to define the amino terminus of Aβ and to generate the soluble amino-terminal fragment β-APP. Subsequent cleavage of the intracellular carboxy-terminal domain of APP by γ-secretase results in the generation of multiple peptides, the two most common being 40-amino acid Aβ (Aβ40) and 42-amino acid Aβ (Aβ42). Amyloid plaques, (invariably associated with Alzheimer's Disease (AD), as well as vascular amyloid deposits in cerebral amyloid angiopathy), contain Aβ, which is believed to play a key role in AD pathophysiology. However, the normal physiological functions of APP still remain unknown.

Beta-secretase (also called BACE; β-site APP-cleaving enzyme) was identified as a membrane-associated aspartyl protease (Vassar, R. et al., Science, 286, 735-741, 1999; Hussain, I. et al., Mol. Cell. Neurosci., 14, 419-427, 1999). BACE mediates the primary amyloidogenic cleavage of βAPP and generates a membrane-bound βAPP C-terminal fragment (APP CTFβ), which is the immediate precursor for the intramembranous γ-secretase cleavage.

Gamma-secretase activity is associated with a protein complex composed of presenilins (PS1 or PS2), nicastrin (Nct), PEN-2, APH-1a, and APH-1b (Yu, G. et al., J. Biol. Chem., 273, 16470-16475, 1998; Capell, A. et al., J. Biol. Chem., 273, 3205-3211, 1998). The expression of these complex components is coordinately regulated, and γ-secretase activity is only detected in the presence of all subunits. The catalytic activity of γ-secretase is most likely contributed by the presenilins. The presenilins are polytopic transmembrane proteins, which together with signal peptide peptidases and type-4 prepilin peptidases may belong to a novel family of aspartyl proteases.

The presenilins, therefore, are essential components of an intramembranous proteolytic activity known as γ-secretase (Wolfe, M. S. et al., J. Biol. Chem., 276, 5413-5416, 2001). Several type-I integral membrane proteins have been identified as substrates for γ-secretase, including the Notch receptor (Notch) and APP (De Strooper, B. et al., Nature (London), 398, 518-522, 1999). Notch is a signaling molecule that regulates cell-fate determination during development (Artavanis-Tsakonas, S. et al., Science, 284, 770-776 1999). Signaling through Notch is triggered by the binding of ligands, such as Delta and Jagged, which induce cleavage of Notch by TACE (Brou, C. et al., Mol. Cell, 5, 207-216, 2000). Subsequent cleavage by γ-secretase releases the Notch intracellular domain, which binds to transcription factors and translocates to the nucleus, where it regulates transcription of Enhancer of Split complex genes (Greenwald, I., Genes Dev., 12, 1751-1762, 1998).

Similarities between the processing of Notch and APP suggests that they may have common functions: the cleavage of APP by γ-secretase liberates a fragment analogous to Notch intracellular domain, the APP intracellular domain (AICD), which could regulate gene expression (Cao, X. et al., Science, 293, 115-120, 2001). AICD has been shown to regulate phosphoinositide-mediated calcium signaling through a γ-secretase-dependent signaling pathway, suggesting that the intramembranous proteolysis of APP may play a signaling role similar to that of Notch (Leissring et al., PNAS, 99, 4697-4702, 2002). It also has been shown that mutations in the presenilins, in addition to their well documented effects of γ-secretase activity, also produce highly consistent alterations in intracellular calcium signaling pathways, which include a potentiation of the phosphoinositide/calcium signaling cascade (Guo, Q. et al., NeuroReport, 8, 379-383, 1996) and deficits in capacitative calcium entry (Leissring, M. A. et al., J. Cell Biol., 149, 793-798, 2000).

Notch signaling has been implicated as a regulatory feature of the angiogenic process (Zhong, T. et al., Nature, 414(6860), 216-220, 2001). Additionally, presenilin knockout mice (mice lacking one or both of the presenilin genes, thus displaying varying degrees of impairment in γ-secretase activity) suffer from abnormal vessel formation (Herreman A. et al., PNAS, 12, 11872-11877, 1999; Shen, J. et al., Cell, 89, 629-639, 1997), suggesting that γ-secretase activity may play a role during angiogenesis.

Thus, there exists a need for compounds that exhibit angiogenesis-inhibiting activity, which can inhibit the pathological angiogenesis observed in cancer and other angiogenic-related diseases, but which have minimal side effects and do not require an extended treatment period and/or combination therapy with other treatment modalities, such as chemotherapy or radiation.

### BRIEF SUMMARY OF THE INVENTION

The present invention provides for the first time the discovery that compounds which inhibit γ-secretase and β-secretase, referred to as γ-secretase and β-secretase inhibitors, exhibit potent anti-angiogenic activity, and that administration of these inhibitors to animals or humans afflicted with disorders associated with pathological angiogenesis, such as cancer, proliferative disorders, or inflammatory disorders, inhibits the pathological angiogenesis observed in the afflicted animals or humans.

In particular, the present invention provides a method of treating tumors or proliferative disorders in animals or humans in need of such treatment by administering to the animal or human therapeutically effective amounts in unit dosage form of a composition containing a carrier and at least one γ-secretase or β-secretase inhibitor that inhibits secretase amyloid precursor protein (APP) processing.

The present invention also provides a method of inhibiting angiogenesis associated with tumors, proliferative or inflammatory disorders in animals or humans in need of such inhibition by administering to the animal or human therapeutically effective amounts in unit dosage form of a composition containing a carrier and at least one γ-secretase or β-secretase inhibitor that inhibits secretase APP processing.

Gamma-secretase inhibitors that are administered according to the method of the present invention can include, without limitation, aspartyl protease transition-state inhibitors, such as L-685,458; dipeptide protease inhibitors, such as DAPT and DAPM; or isocoumarin-based serine protease inhibitors, such as JLK-6.

Beta-secretase inhibitors that are administered according to the method of the present invention can include, without limitation, peptidomimetic tight binding transition-state analogue inhibitors, such as OM99-2, or substrate analogue peptide inhibitors, such as Z-VLL-CHO GL 189, or P10-P4′statV.

Tumors that can be treated according to the method of the present invention include, without limitation, malignant brain tumors, such as glioblastomas; malignant lung tumors, such as adenocarcinomas; or malignant tumors of the breast, colon, kidney, bladder, head or neck. Proliferative disorders that can be treated according to the method of the present invention include, without limitation, hematopoietic disorders, such as leukemias, lymphomas or polycythemias; ocular disorders, such as diabetic retinopathy, macular degeneration, glaucoma or retinitis pigmentosa. Inflammatory disorders that can be treated according to the method of the present invention include, without limitation, rheumatoid arthritis, osteoarthritis, pulmonary fibrosis, sarcoid granulomas, psoriasis or asthma.

### BRIEF DESCRIPTION OF THE DRAWINGS

FIG. 1a: Effect of β and γ secretase inhibitors on the viability of human brain endothelial cells. The potential toxicity of various doses of β and γ secretase inhibitors was estimated by measuring LDH activity in the culture medium. ANOVA revealed no main effect for L-685,458 but a significant main effect for Z-VVL-CHO (P<0.03), OM99-2 (P<0.006) and DAPT (P<0.009). Post-hoc analysis did not show any significant difference between control and any of the β and γ secretase inhibitors tested (P>0.05) showing that the β and γ secretase inhibitors did not induce endothelial cell death at the doses employed.

FIG. 1b: Effect of β and γ secretase inhibitors on the proliferation of human brain endothelial cells. The amount of viable cells following 24 hours of treatment with various doses of b and g secretase inhibitors was measured using the Quick cell proliferation assay kit. ANOVA revealed a significant main effect of L-685,458 dose (P<0.001), of Z-VVL-CHO dose (P<0.001), of OM99-2 dose (P<0.001) and of DAPT (P<0.001). Post-hoc analysis showed significant differences between control and 2 μM L-685,458 (P<0.001), between control and 5 μM Z-VVL-CHO (P<0.001), control and 5 μM OM99-2 (P<0.001) and between control and 5 μM DAPT (P<0.001).

FIG. 2a: Representative pictures showing the effect of L-685,458 and Z-VVL-CHO on capillary morphogenesis.

FIG. 2b: Quantification of network length by Image analysis. The numbers in parenthesis on the x-axis represent the number of 4X fields analyzed. ANOVA revealed significant main effects of Z-VVL-CHO (P<0.001) and L-685,458. Post-hoc testing showed significant difference between control and Z-VVL-CHO for all the doses tested (P<0.001) and between control and L-685,458 for all the doses tested (P<0.001).

FIG. 3a-b: Representative pictures showing the effect of various DAPT doses and of various OM99-2 doses on capillary morphogenesis.

FIG. 3c: Quantification of network length by Image analysis. The numbers in parenthesis on the x-axis represent the number of 4X fields analyzed. ANOVA revealed significant main effects of DAPT dose and OM99-2 dose (P<0.001). Post-hoc testing showed significant difference between control and DAPT 1 μM (P<0.005), control and OM99-2 1 μM (P<0.005), control and DAPT 5 μM (P<0.001), control and OM99-2 2 μM (P<0.001) and between control and OM99-2 5 μM (P<0.001).

FIG. 4a-c: Effects of β and γ secretase inhibitors on the metabolism of APP in human brain endothelial cells. FIG. 4a: Media were analyzed by immunoblotting to measure secreted α-sAPP molecules. FIG. 4b: Carboxyl-terminal APP fragments and full length APP from human brain endothelial cell lysates. FIG. 4c: Quantification of α-sAPP molecules secreted in the culture media of human brain endothelial cells. ANOVA revealed a significant main effect for Z-VVL-CHO (P<0.003) but not for L-685,458 (P=0.13) and post-hoc analysis showed significant differences between control and Z-VVL-CHO (P<0.006) showing that Z-VVL-CHO significantly inceases the secretion of α-sAPP. Quantification of carboxyl-terminal APP fragments generated by human brain endothelial cells. ANOVA revealed significant main effects of OM99-2 (P<0.002), L-685,458 (P<0.001) and DAPT (P<0.001). Post-hoc testing showed significant differences between control and OM99-2 (P<0.009), control and DAPT (P<0.001) and between control and L-685,458 (P<0.001) showing that DAPT and L-685,458 stimulates whereas OM99-2 significantly reduces the accumulation of carboxyl-terminal APP fragments.

FIG. 5a-b: The β-secretase inhibitor Z-VVL-CHO dose-dependently inhibits the formation of microvessel outgrowths by explants of rat aortae. FIG. 5a: Representative pictures of rat aortic rings embedded in Matrigel showing the progressive sprouting of capillaries with time in function of the dose of Z-VVL-CHO used. FIG. 5b: Quantification by image analysis of the area covered by microvessel outgrowths. ANOVA revealed a significant main effect of Z-VVL-CHO dose (P<0.001) and time (P<0.001) as well as an interactive term between them (P<0.001). Post-hoc testing showed significant differences between control and 100 nM Z-VVL-CHO (P<0.001), control and 1 μM Z-VVL-CHO (P<0.001) and between control and 5 μM Z-VVL-CHO (P<0.001).

FIG. 6a-b: Effect of the β-secretase inhibitors OM99-2 and P10-P4′statV on the sprouting of microvessels by explants of rat aortae. FIG. 6a: Representative pictures showing the formation of microvessel outgrowths in function of time for control aortic rings, for aortic rings treated with 20 μM of P10-P4′statV and aortic rings treated with 20 μM of OM99-2. FIG. 6b: Quantification by image analysis of the area covered by microvessel outgrowths. ANOVA revealed significant main effects for P10-P4′statV (P<0.001) and for OM99-2 dose (P<0.001) as well as an interactive term between time and P10-P4′statV (P<0.002) and between time and OM99-2 dose (P<0.002). Post-hoc testing across day 5 and day 6 showed significant differences between control and P10-P4′statV (P<0.001), control and 1 μM OM99-2 (P<0.003), control and 5 μM OM99-2 (P<0.001) and between control and 20 μM OM99-2 (P<0.001).

FIG. 7a-c: Effects of γ-secretase inhibitors on the formation of microvessel outgrowths by explants of rat aortae. FIG. 7a: Representative pictures depicting the effect of DAPT on microvessel outgrowths. FIG. 7b: Quantification by image analysis of the area covered by microvessel outgrowths following DAPT treatment. ANOVA revealed significant main effects of DAPT dose (P<0.001) and time (P<0.001) as well as an interactive term between them (P<0.001). Post-hoc testing showed significant differences between control and DAPT 5 μM (P<0.001), between control and DAPT 20 μM (P<0.02) but no significant difference between control and DAPT 10 μM (P=0.999). FIG. 7c: Quantification by image analysis of the area covered by microvessel outgrowths following L685,458 treatment. ANOVA revealed significant main effects of L685,458 (P<0.001) and time (P<0.001) as well as an interactive term between them (P<0.005). Post-hoc testing showed significant differences between control and 1 μM L685,458 (P<0.002) and between control and 5 μM L685,458 (P<0.001).

FIG. 8a: Anti-tumoral effect of the γ-secretase inhibitor DAPT and of the β-secretase inhibitor Z-VLL-CHO on human glioblastoma (U-87 MG) xenografts growth rates. U-87 MG cells (6×10⁶) were injected subcutaneously into both flanks of 8-10 weeks-old nude mice. Mice were injected intraperitoneally with either the vehicle, 5 mg/Kg of body weight of the β-secretase inhibitor Z-VLL-CHO or 10 mg/Kg of body weight of the γ-secretase inhibitor DAPT, starting when tumors had reached a mean tumor volume of approximately 140 mm³ (day 8 post implantation). Injections were given everyday for 9 days. Data are expressed as mean tumor volume±S.E. ANOVA reveals significant main effect of DAPT (P<0.001) and Z-VLL-CHO (P<0.001) and time (P<0.001). Post-hoc analysis shows significant differences between the tumor volumes from vehicle treated mice and DAPT treated animals (P<0.001), from vehicle treated mice and Z-VLL-CHO treated mice (P<0.001) but no difference between Z-VLL-CHO and DAPT treatments (P=0. 12), showing that Z-VLL-CHO and DAPT inhibit the growth of human glioblastoma xenografts with a similar potency. FIG. 8b: Representative pictures of sections of glioblastoma tumors immunostained with CD31 antibodies. FIG. 8c: Histogram depicting the estimation of glioblastoma tumor vascularization. ANOVA reveals significant main effects of DAPT ( P<0.001) and Z-VLL-CHO (P<0.02). Post-hoc analysis shows significant differences between the vascular index of vehicle treated mice and DAPT treated mice (P<0.002) and between the vascular index of vehicle treated mice and Z-VLL-CHO treated animals (P<0.03) showing that Z-VLL-CHO and DAPT significantly reduce the vascularization of human glioblastoma xenografts.

FIG. 9a: Anti-tumoral effect of the γ-secretase inhibitor DAPT and of the β-secretase inhibitor Z-VLL-CHO on human lung adenocarcinoma (A-549) xenografts growth rates. A-549 cells (8.5×10⁶) were injected subcutaneously into both flanks of 8-10 weeks-old nude mice. Mice were injected intraperitoneally with either the vehicle, 5 mg/Kg of body weight of the β-secretase inhibitor Z-VLL-CHO or 10 mg/Kg of body weight of the γ-secretase inhibitor DAPT, starting when tumors had reached a mean tumor volume of approximately 200 mm³ (day 21 post implantation). Injections were given everyday for 12 days. Data are expressed as mean tumor volume±S.E. ANOVA reveals significant main effects of DAPT (P<0.001), Z-VLL-CHO (P<0.001) and time (P<0.001). Post-hoc analysis shows significant differences between the tumor volumes from vehicle treated mice and DAPT treated animals (P<0.001), from vehicle treated mice and Z-VLL-CHO treated mice (P<0.001) but no difference between Z-VLL-CHO and DAPT treatments (P=0.519), showing that DAPT and Z-VLL-CHO significantly inhibit the growth of human lung adenocarcinoma xenografts. FIG. 9b: Representative pictures of sections of lung adenocarcinoma tumors immunostained with CD31 antibodies. FIG. 9c: Histogram depicting the quantification of the vascularization of lung adenocarcinoma tumors. ANOVA reveals significant main effects of DAPT (P<0.01) and Z-VLL-CHO (P<0.01). Post-hoc analysis shows significant differences between the vascular index of vehicle treated mice and DAPT treated mice (P<0.03) and between the vascular index of vehicle treated mice and Z-VLL-CHO treated animals (P<0.03), showing that Z-VLL-CHO and DAPT significantly reduce the vascularization of human lung adenocarcinoma xenografts.

FIG. 10: Anti-tumoral effect of the γ-secretase inhibitor JLK-6 on human lung adenocarcinoma (A-549) xenografts growth rates. A-549 cells (8.5×10⁶) were injected subcutaneously into both flanks of 8-10 weeks-old nude mice. Mice were injected intraperitoneally everyday from Day 16 (when the tumors reached a volume of approximately 150 mm³) with either the vehicle or 5 mg/Kg of body weight of the γ-secretase inhibitor JLK-6. Data are expressed as mean tumor volume (mm³)±S.E. ANOVA reveals significant main effects of JLK-6 (P<0.001) and time (P<0.001), showing inhibition of tumor growth in the JLK-6 treated group of animals compared to animals treated with the vehicle, showing that JLK-6 significantly inhibits the growth of human lung adenocarcinoma xenografts.

### DESCRIPTION OF THE PREFERRED EMBODIMENTS

The present invention provides methods for treating tumors or proliferative disorders in animals or humans in need of such treatment, comprising the administration of therapeutically effective amounts in unit dosage form of a composition comprised of a carrier and at least one γ-secretase or β-secretase inhibitor that inhibits γ-secretase or β-secretase APP processing.

The present invention also provides methods for inhibiting angiogenesis in animals or humans in need of such inhibition, comprising the administration of therapeutically effective amounts in unit dosage form of a composition comprised of a carrier and at least one γ-secretase or β-secretase inhibitor that inhibits γ-secretase or β-secretase APP processing.

The γ-secretase inhibitors administered according to the method of the present invention can include, without limitation, aspartyl protease transition state analogue γ-secretase inhibitors, having a general backbone structure, illustrated below. (“R” refers to analogue substitutions.)

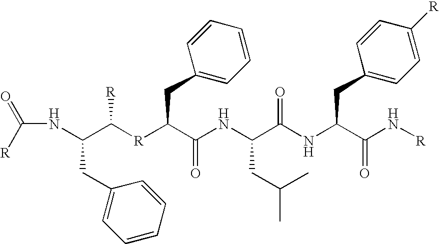

In particular, an aspartyl protease transition state analogue γ-secretase inhibitor administered according to the method of the present invention is L-685,458 ({1S-Benzyl-4R-[1-(1S-carbamoyl-2-phenethylcarbamoyl)-1S-3-methylbutylcarba-moyl]-2R-hydroxy-5-phenyl-pentyl}carbamic acid tert-butyl ester), a cell-permeable hydroxyethylene dipeptide isostere that acts as a highly specific and potent inhibitor of γ-secretase (Abₜₒₜₐₗ IC₅₀=17 nM, Ab₄₀ IC₅₀=48 nM, and Ab₄₂ IC₅₀=67 nM). L-685,458 functions as a transition state analogue at the catalytic site of an aspartyl protease, and exhibits a similar potency toward Aβ40 and Aβ42. L-685,458 exhibits over a hundred-fold greater selectivity for γ-secretase than for the aspartyl protease cathepsin. L-685,458 also binds to presenilin and blocks Notch intracellular domain production.

Another class of γ-secretase inhibitors administered according to the method of the present invention can include, without limitation, dipeptide protease γ-secretase inhibitors having the general backbone structures illustrated below. (R refers to analogue substitutions.)

In particular, DAPM (N-[N-3,5-difluorophenacetyl]-L-alanyl-S-phenylglycine

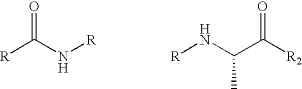

methyl ester), is a cell permeable, dipeptide protease inhibitor of γ-secretase (IC₅₀ Ab˜10 nM in 7PA2 cells) with anti-aggregation properties. DAPM prevents early Aβ oligomerization by selectively blocking Aβ dimer and trimer formation. Another dipeptide protease γ-secretase inhibitor is DAPT (N-[N-(3,5-difluorophenacetyl-L-alanyl)]-S-phenylglycine t-butyl ester).

Still another class of γ-secretase inhibitors administered according to the method of the present invention can include isocoumarin-based serine protease γ-secretase inhibitors, having the general backbone structure illustrated below. (R refers to analogue substitutions.)

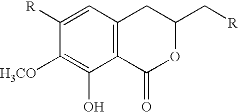

In particular, an isocoumarin-based serine protease γ-secretase inhibitor administered according to the method of the present invention is JLK-6 (7-amino-4-chloro-3-methoxyisocoumarin), a cell-permeable, active site-directed, irreversible serine protease γ-secretase inhibitor that belongs to the class of isocoumarin analogues. JLK-6 acts as a potent and selective inhibitor of γ-secretase and blocks the production of both Aβ40 and Aβ42 (IC₅₀<100 mM) in HEK293 cells expressing wild-type and Swedish-mutant APP. Additionally, JLK-6 does not affect either the processing of Notch or the proteolysis of presenilin 1 and 2.

The β-secretase inhibitors administered according to the method of the present invention can include, without limitation, peptidomimetic tight binding transition-state analogue β-secretase inhibitors, which all contain a similar peptidomimetic structural backbone, illustrated below. (“R” refers to analogue substitutions.)

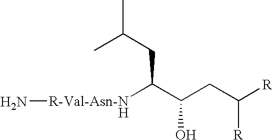

In particular, OM99-2 (Glu-Val-Asn-Leu-ψ-Ala-Ala-Glu-Phe [ψ denotes replacement of CONH by (S)—CH(OH)CH₂]) is an aspartyl protease inhibitor that acts as a peptidomimetic tight binding transition-state analogue β-secretase inhibitor. OM99-2 is designed from the template of the β-secretase site of Swedish APP with an Asp to Ala replacement. The OM99-2 compound also includes a nonhydrolyzable hydroxyethylene isostere between the amino acids leucine and alanine (above-described ψ replacement).

Another peptidomimetic β-secretase inhibitor, Z-VLL-CHO (N-benzyloxycarbony-val-leu-leucinal), is a peptide aldehyde protease inhibitor that exhibits potent, cell-permeable and reversible inhibition of β-secretase. Z-VLL-CHO corresponds to the β-secretase cleavage site (VNL-DA) of the Swedish mutant APP, and inhibits the formation of both Aβₜₒₜₐₗ (IC₅₀=700 nM) and Aβ42 (IC₅₀=2.5 μM) in Chinese hamster ovary cells stable transfected with wild-type APP751.

Two other peptidomimetic β-secretase inhibitors, GL189 (H-Glu-Val-Asn-Statine-Val-Ala-Glu-Phe-NH) and P10-P4′statV (H-Lys-Thr-Glu-Glu-Ile-Ser-Glu-Val-Asn-Stat-Val-Ala-Glu-Phe-OH [Stat=(3S,4S)-Statine]), are substrate analogue BACE inhibitors. GL189 completely blocks the proteolytic activity (at 5 μM) of β-secretase in solubilized membrane fractions from BACE transfected MDCK cells, and P10-P4′statV (H-Lys-Thr-Glu-Glu-Ile-Ser-Glu-Val-Asn-Stat-Val-Ala-Glu-Phe-OH [Stat=(3S,4S)-Statine]) is a potent inhibitor of APP protein (IC₅₀=30 nM). Stat refers to the unusual amino acid statine ((3S,4S)-4-amino-3-hydroxy-6-methylheptanoic acid), which has become a prototypical hydroxymethylene isostere, and is contained in pepstatin, the naturally occurring peptide produced by various Streptomyces species.

Examples of “R” analogue substitutions include, without limitation, H, OH, CH₃ and OCH₃. R can be greatly variable as known in the art.

Examples of tumors that can be treated according to the method of the present invention include, without limitation, malignant brain tumors, such as glioblastomas; malignant lung tumors, such as adenocarcinomas; or malignant tumors of the breast, colon, kidney, bladder, head or neck. Proliferative disorders that can be treated according to the method of the present invention include, without limitation, hematopoietic disorders, such as leukemias, lymphomas or polycythemias; and ocular disorders, such as diabetic retinopathy, macular degeneration, glaucoma or retinitis pigmentosa. Inflammatory disorders that can be treated according to the method of the present invention include, without limitation, rheumatoid arthritis, osteoarthritis, pulmonary fibrosis, sarcoid granulomas, psoriasis or asthma.

Compositions containing γ-secretase or β-secretase inhibitors can be administered to a patient via various routes including parenterally, orally or intraperitoneally. Parenteral administration includes the following routes: intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, or nasal inhalation via insufflation or nebulization.

Compounds containing γ-secretase or β-secretase inhibitors that are orally administered can be enclosed in hard or soft shell gelatin capsules, or compressed into tablets. Compounds also can be incorporated with an excipient and used in the form of ingestible tablets, buccal tablets, troches, capsules, sachets, lozenges, elixirs, suspensions, syrups, wafers, and the like. Compositions containing γ-secretase or β-secretase inhibitors can be in the form of a powder or granule, a solution or suspension in an aqueous liquid or non-aqueous liquid, or in an oil-in-water or water-in-oil emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain the active compound, sucrose as a sweetening agent, methyl and propylparabens as preservatives, a dye and flavoring. Any material used in preparing any dosage unit form should be pharmaceutically pure and substantially non-toxic. Additionally, the γ-secretase or β-secretase inhibitors can be incorporated into sustained-release preparations and formulations.

The γ-secretase or β-secretase inhibitors can be administered to the CNS, parenterally or intraperitoneally. Solutions of the compound as a free base or a pharmaceutically acceptable salt can be prepared in water mixed with a suitable surfactant, such as hydroxypropylcellulose. Dispersions also can be prepared glycerol, liquid polyethylene glycols, and mixtures thereof, and in oils. Under ordinary conditions of storage and use, these preparations can contain a preservative and/or antioxidants to prevent the growth of microorganisms or chemical degeneration.

The pharmaceutical forms suitable for injectable use include, without limitation, sterile aqueous solutions or dispersions and sterile powders for the extemporaneous preparation of sterile injectable solutions or dispersions. In all cases, the form must be sterile and must be fluid to the extent that easy syringability exists. It can be stable under the conditions of manufacture and storage and must be preserved against the contaminating action of microorganisms, such as bacteria and fungi. The carrier can be a solvent or dispersion medium which contains, for example, and without limitation, water, ethanol, polyol (such as glycerol, propylene glycol, and liquid polyethylene glycol), suitable mixtures thereof, or vegetable oils. The proper fluidity can be maintained, for example, by the use of a coating, such as lecithin, by the maintenance of the required particle size (in the case of a dispersion) and by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars or sodium chloride.

Sterile injectable solutions are prepared by incorporating the γ-secretase or secretase inhibitor(s) in the required amount in the appropriate solvent with various of the other ingredients enumerated above, as required, followed by filtered sterilization. Generally, dispersions are prepared by incorporating the various sterilized γ-secretase or β-secretase inhibitor(s) into a sterile vehicle that contains the basic dispersion medium and any of the other ingredients from those enumerated above. In the case of sterile powders for the preparation of sterile injectable solutions, the preferred methods of preparation are vacuum drying and freeze drying.

Pharmaceutical compositions which are suitable for administration to the nose or buccal cavity include, without limitation, self-propelling and spray formulations, such as aerosol, atomizers and nebulizers.

The therapeutic γ-secretase or β-secretase inhibitors of the present invention can be administered to an animal or human alone or in combination with pharmaceutically acceptable carriers or as pharmaceutically acceptable salts, the proportion of which is determined by the solubility and chemical nature of the compound, chosen route of administration, and standard pharmaceutical practice. Examples of animals include, without limitation, mammalian and non-mammalian animals and vertebrates and invertebrates.

It has now been demonstrated that γ-secretase and β-secretase inhibitors dose dependently affect the proliferation and differentiation of human brain endothelial cells into capillaries, the formation of microvessel outgrowths in a rat aortic ring model of angiogenesis, and the growth and vascularization of human lung adenocarcinomas. This suggests, without being bound by any particular theory, that γ-secretase and β-secretase activities are required during the angiogenic process. Among the inhibitors tested, the β-secretase inhibitor Z-VLL-CHO appears to be profoundly anti-angiogenic, both in a capillary morphogenesis assay and in a rat aortic ring model of angiogenesis. Additionally, it has now been demonstrated that the above-identified γ-secretase and β-secretase inhibitors can completely inhibit the growth of human brain and human lung adenocarcinoma tumors xenografted into nude mice, which are dependent on angiogenesis for their growth. Finally, the γ-secretase inhibitor, JLK-6, appears to reduce angiogenesis in vitro and to inhibit the growth of human lung tumor xenografts into nude mice, suggesting that the anti-angiogenic activity of γ-secretase inhibitors are independent of Notch cleavage, because this γ-secretase inhibitor does not affect Notch processing. It is believed, without being bound by any particular theory, that in both human brain and human lung tumor models, the anti-tumor activity of the γ-secretase and β-secretase inhibitors is mediated by inhibition of angiogenesis, because microvessel density values in the treated tumors have been shown to be significantly decreased.

The following non-limiting examples describe in more detail the effects of γ-secretase and β-secretase inhibitors on the proliferation and differentiation of human brain endothelial cells, capillary morphogenesis, processing of APP in human brain endothelial cells, micro-vessel sprouting, and brain and lung tumor growth.

### EXAMPLE 1

### Effect of β-Secretase and γ-Secretase Inhibitors on Capillary Morphogenesis

An investigation was undertaken to determine the effects of the aspartyl protease transition-state γ-secretase inhibitor L-685,458; the dipeptide protease γ-secretase inhibitors DAPM and DAPT; the isocoumarin-based serine protease γ-secretase inhibitor JLK-6; the substrate analogue peptide β-secretase inhibitors Z-VLL-CHO and GLI89; and the peptidomimetic tight binding transition-state analogue β-secretase inhibitor OM99-2, on the proliferation and differentiation of primary cultures of human brain endothelial cells, on capillary morphogenesis, and on the processing of APP in human brain endothelial cells, in order to determine the potential role of the APP processing pathway in angiogenesis. Additionally, the processing of APP in human brain endothelial cells was investigated by determining the effects of the above-identified γ-secretase and β-secretase inhibitors on α-sAPP secretion and on the production of intracellular APP carboxyl-terminal fragments (CTF) in primary cultures of human brain endothelial cells.

### Materials and Methods

1. Isolation and Culture of Endothelial Cells from Microvessel Outgrowths.

Matrigel-containing microvessel outgrowths from human middle cerebral arteries isolated following rapid autopsies (2 to 4 hours post-mortem delay) were dissected with the aid of an inverted microscope and dissociated several times in endothelial basal medium (EBM) through a sterile pipette tip. Matrigel fragments were then plated on plastic culture flasks, and incubated in EBM (supplemented with 2% fetal bovine serum and 1×penicillin-streptomycin-fungizone mixture) at 37° C., 5% CO₂ with medium changed every 3 days. After 5 to 6 days in culture, cells were subjected to a double immunostaining with an antibody against factor VIII and an antibody against α-smooth muscle actin in order to verify their endothelial nature.

2. Measurement of Human Brain Endothelial Cells Viability and Proliferation.

Primary cultures of human brain endothelial cells were plated at a density of 104 cells/200 mL of EBM 4% in 96 wells culture plates and treated with various doses of γ-secretase and β-secretase inhibitors as indicated in the figure legends. Following 24 hours in culture, the EBM covering the cells was removed and assayed for Lacticodehydrogenase (LDH) activity using the cytotoxicity detection kit (Roche Diagnostic Corporation, Ind.). Cells were covered with 100 mL of EBM 4% and cellular proliferation measured using the Quick cell proliferation assay kit (Biovision Research Products, Calif.).

3. Capillary Morphogenesis Assay.

Two hundred μl of Matrigel were placed into each well of a 24-well culture plate at 4° C. and allowed to polymerize by incubation at 37° C. Human middle cerebral artery endothelial cells (5×104) were seeded on the Matrigel in 1 ml of EBM containing 4% fetal calf serum. The cells were incubated at 37° C. for 20 hours in a humidified 5% CO₂ atmosphere in the presence or absence of various doses of γ-secretase and β-secretase inhibitors as indicated in the figure legends. The experiments were performed in quadruplicate for each treatment condition. For each culture, 2 randomly chosen fields were photographed using a 4×objective. An experimenter unaware of the different treatments measured the total length of tube structures in each photograph using the Image Pro Plus software (Media Cybernetic, Inc., Md.). Capillary network lengths for the different treatment conditions were expressed as the percentage of capillary network lengths obtained in the control condition.

4. Alpha-sAPP Immunoprecipitation, SDS PAGE and Immunoblotting

Confluent human brain endothelial cells (grown on 75 cm2 flasks with EBM 4% FBS medium) were treated for 24 hours with 5 μM of Z-VVL-CHO, 5 μM of L-685,458, 5 μM of OM99-2, 5 AM of DAPT or went untreated (control). Experiments were done in quadriplicate for each treatment condition. 6E10 (Signet), a mAb that recognizes residues 1-17 of human Aβ (Van Nostrand et al., Nature, 341, 546-549, 1989) was used to immunoprecipitate soluble APP generated following cleavage by α-secretase from cell culture medium. Immunoprecipitated material was resolved on a 4-20% gradient SDS-PAGE, transferred to PVDF membranes and immunodetected with mAb 22C11 (Roche Diagnostics) that recognizes the amino acids 66-81 of the N-terminal portion of APP (Hibich, C., J. Biol. Chem., 268, 26571-26577, 1993). Human brain endothelial cells were lysed on ice using MPERTM reagent (Pierce) supplemented with 1 mM PMSF and 1 mM of sodium-orthovanadate. Samples were sonicated and centrifuged at 10,000 g for 30 min at 4° C. The protein content of the lysates was determined using the BCA Protein assay kit (Pierce). Total lysates (50 mg of protein/sample) were separated on a 4-20% gradient SDS-PAGE and transferred to PVDF membranes and immunoprobed with mAb 22C11 in order to detect full length APP and also immunoprobed with an Anti-APP-CT20 (Calbiochem) antibody which recognizes the amino acid residues 751-770 of the carboxyl terminal region of APP (Pinnix et al. 2001; J. Biol. Chem., 276, 481).

### Results

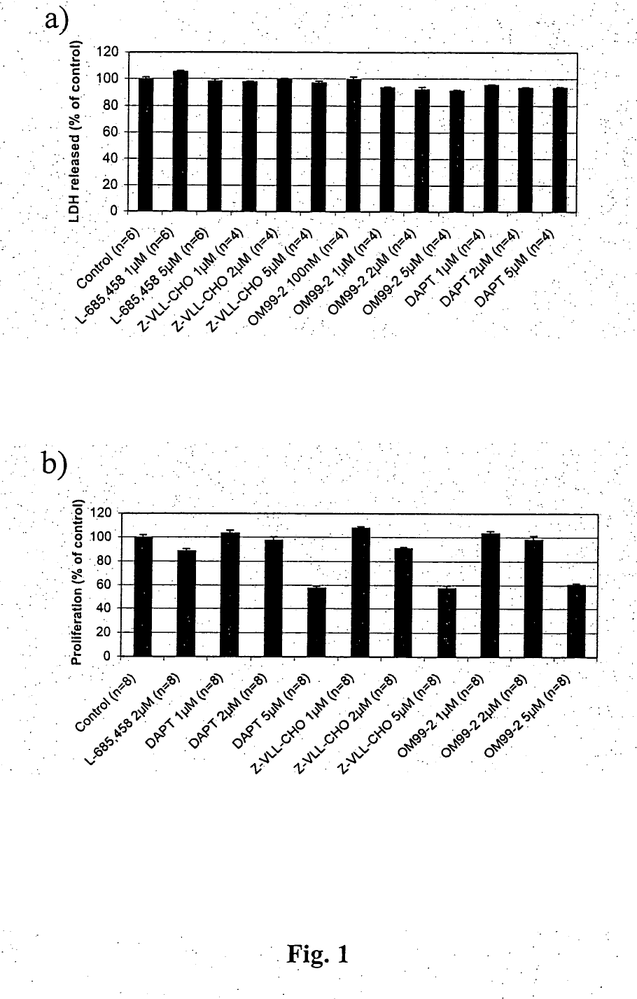

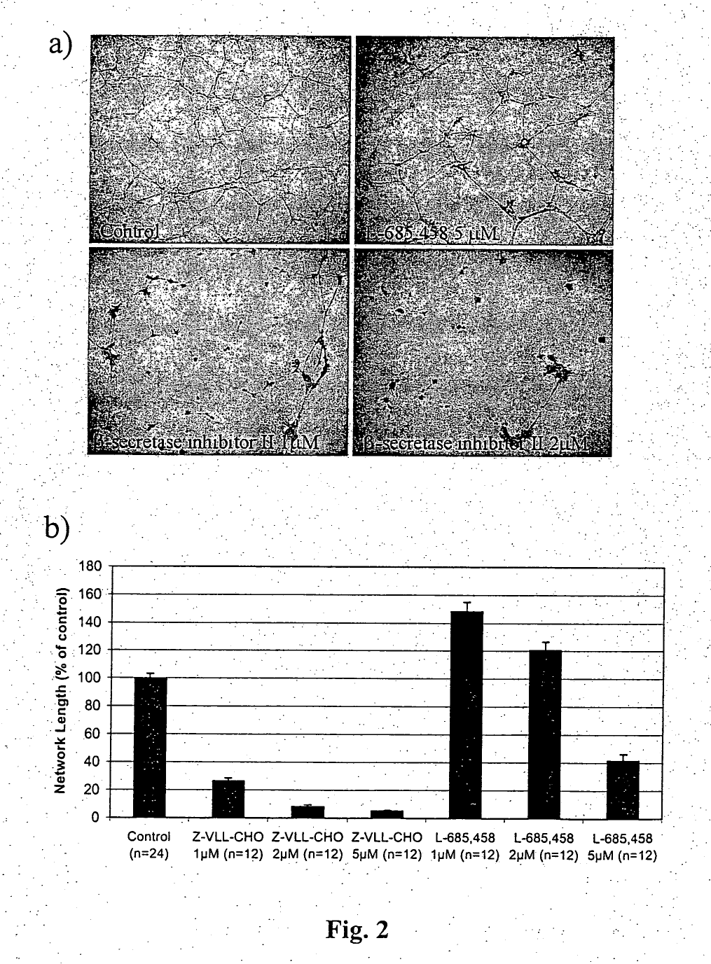

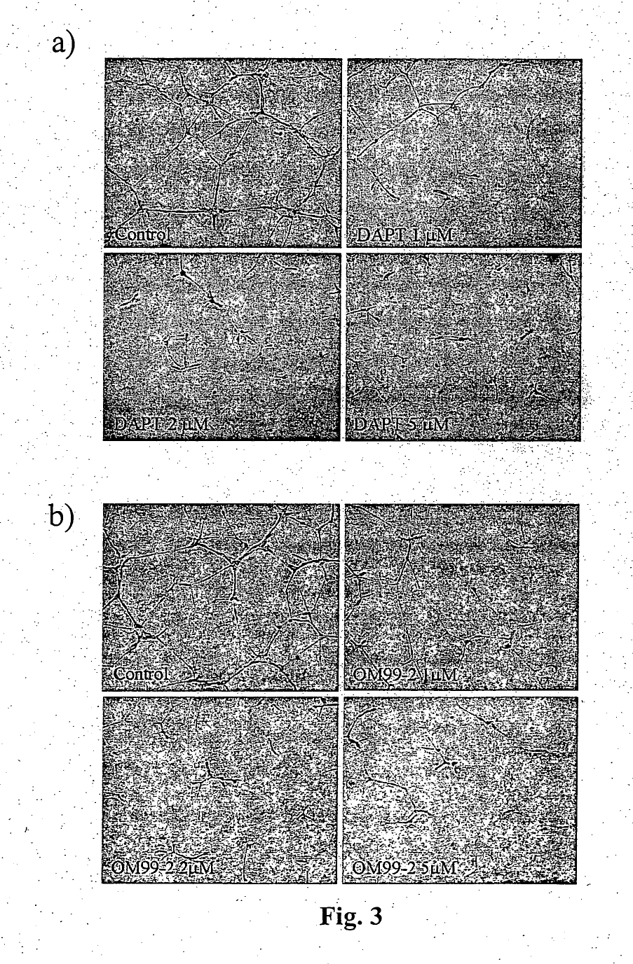

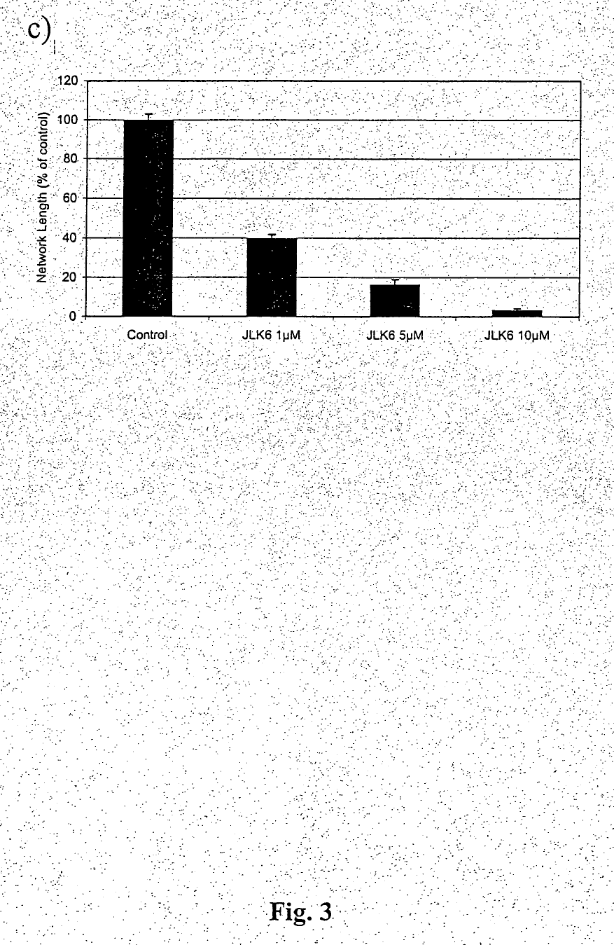

The γ-secretase inhibitors, L-685,458, DAPM, DAPT and JLK-6, all dose dependently inhibited the proliferation of human brain endothelial cells without inducing cellular toxicity (FIG. 1 and 3). When plated on a reconstituted basement membrane, endothelial cells ceased proliferating and differentiating into a network of capillary structures. In particular, at a low dose, L-685,458 actually stimulated capillary morphogenesis whereas a 5 μM dose of the inhibitor appeared to have a potent angiogenic effect (FIG. 2), suggesting that a γ-secretase-like activity is required during the angiogenic process.

The β-secretase inhibitors, Z-VLL-CHO, OM99-2 and GL189 all dose dependently inhibited the proliferation of human brain endothelial cells without affecting their viability (FIG. 1 and 3). Additionally, these β-secretase inhibitors also potently and dose dependently inhibited the formation of capillary structures in the capillary morphogenesis assay (FIG. 2), suggesting that β-secretase activity also contributes to the angiogenic process.

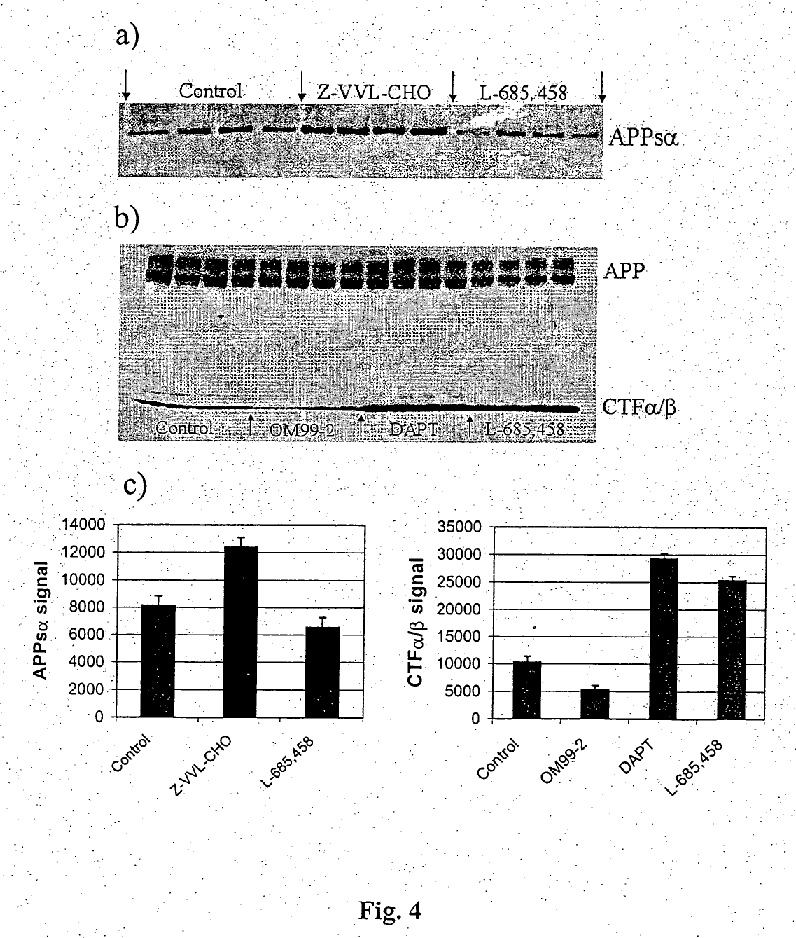

The β-secretase inhibitor, Z-VLL-CHO, stimulated the secretion of α-sAPP, suggesting an inhibition of β-secretase activity. The γ-secretase inhibitors, DAPT AND L-685-485 promoted the accumulation of APP CTF in human brain endothelial cells, modeling the accumulation of APP CTF habitually observed in PSI knockout cells deficient in γ-secretase activity (FIG. 4).

### EXAMPLE 2

### Effect of γ-Secretase and β-Secretase Inhibitors on the Sprouting of Microvessels from Explants of Rat Aortae

An investigation was undertaken to determine the effects of the aspartyl protease transition-state γ-secretase inhibitor L-685,458; the dipeptide protease γ-secretase inhibitors DAPM and DAPT; the isocoumarin-based serine protease γ-secretase inhibitor JLK-6; the substrate analogue peptide β-secretase inhibitors Z-VLL-CHO, GL189 and P10-P4′statV; and the peptidomimetic tight binding transition-state analogue β-secretase inhibitor OM99-2, on the rat aortae model of angiogenesis, which is known to correlate well with in vivo events of neovascularization. In this assay, angiogenesis is a self-limited process, triggered by injury and regulated by well-defined autocrine-paracrine mechanisms (Nicosia et al., Amer. J. Path., 151, 1379-1385, (1997). When the rat aortic endothelium is exposed to a three-dimensional matrix, it switches to a microvascular phenotype that generates branching networks of microvessels (Nicosia et al., Atherosclerosis, 95, 191-199,1992).

### Materials and Methods

Twenty four well tissue culture grade plates (Nalgene International, NY) were covered with 250 mL of Matrigel (Becton-Dickinson, Bedford, Mass.) and allowed to gel for 30 min at 37° C., 5% CO2. Briefly, thoracic aortae were excised from 9 month-old Sprague Dawley rats. After removing the fibroadipose tissue, arteries were sectioned into 1 mm long cross sections, rinsed 5 times with EBM (Clonetics Corp.) containing 4% fetal bovine serum (FBS) and placed on the Matrigel coated wells. Artery rings were covered with an additional 250 mL of Matrigel. After polymerization the Matrigel was covered with 1 mL of EBM (4% FBS) containing various doses of Z-VVL-CHO, OM99-2, P10-P4′statV, DAPT, or L-685,458 as indicated in the figure legends (the culture medium was changed every 3 days). Pictures were taken at day 4, 5 and 6 using a 4×objective. Microvessel outgrowth area was quantified using the Image Pro Plus software. Briefly, ring cultures were photographed using a digital video camera linked to an Olympus BX60 microscope. The outgrowth area was delineated and measured with the Image Pro Plus software by using a strategy of microvessel outgrowth detection based on difference in color intensities between the outgrowths, the Matrigel and the artery ring. The artery rings were manually selected and excluded from the area of measurement and the color intensity threshold was adjusted to selectively measure the area occupied by the microvessel outgrowths. Results were expressed as a percentage of the area occupied by microvessel outgrowths at day 4 in control condition.

### Results

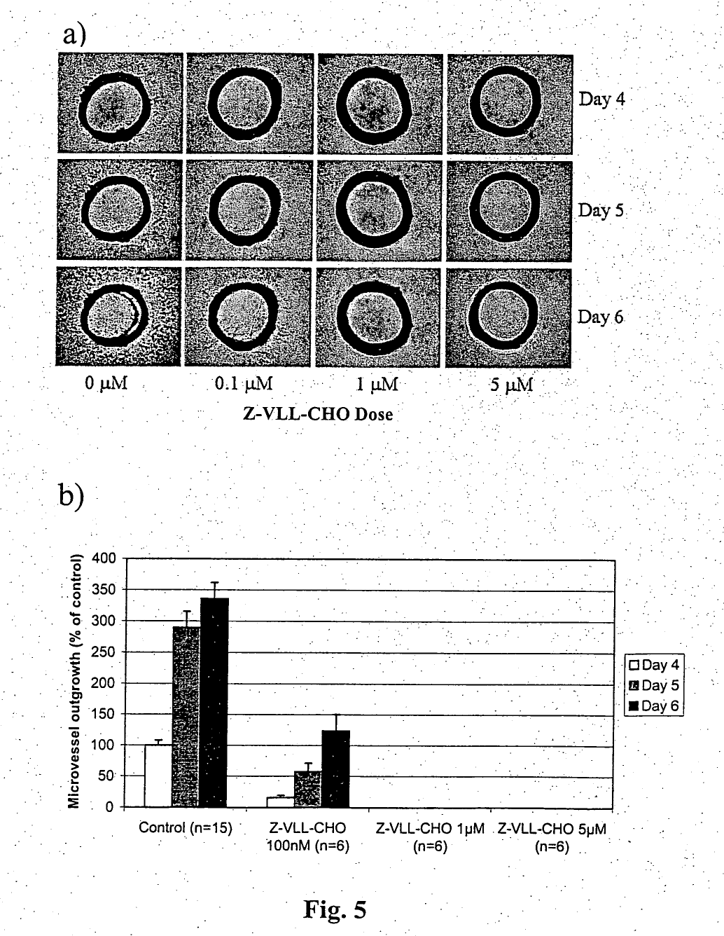

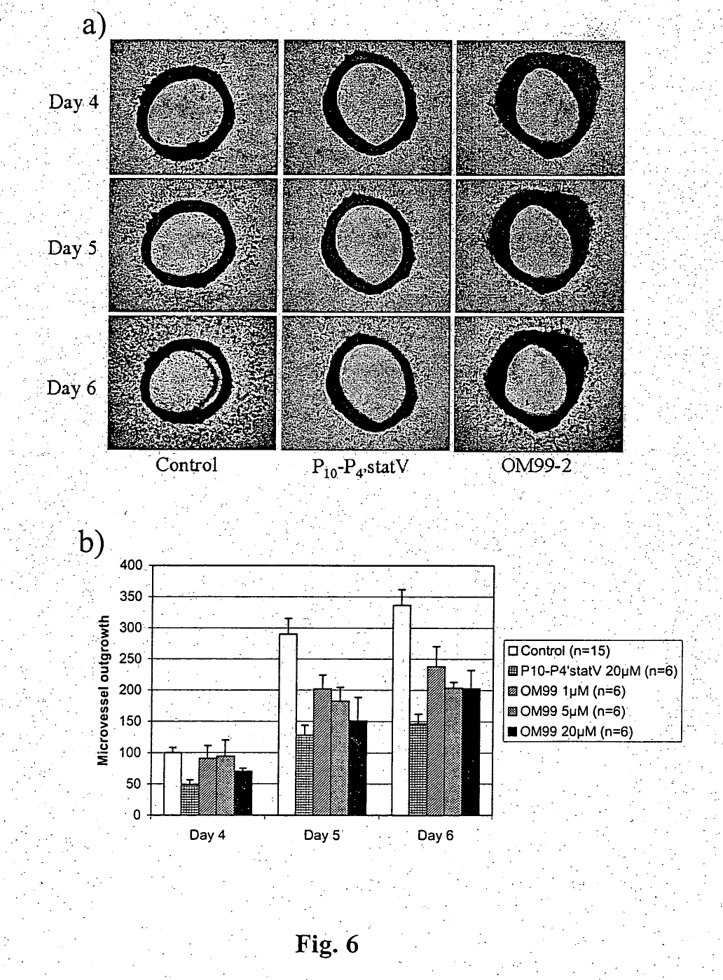

The β-secretase inhibitors, Z-VLL-CHO, OM99-2 and P10-P4′stat all dose dependently and potently inhibited the sprouting of microvessel outgrowths from explants of rat aortae (FIG. 5-6), suggesting the involvement of β-secretase-like activity during the angiogenic process.

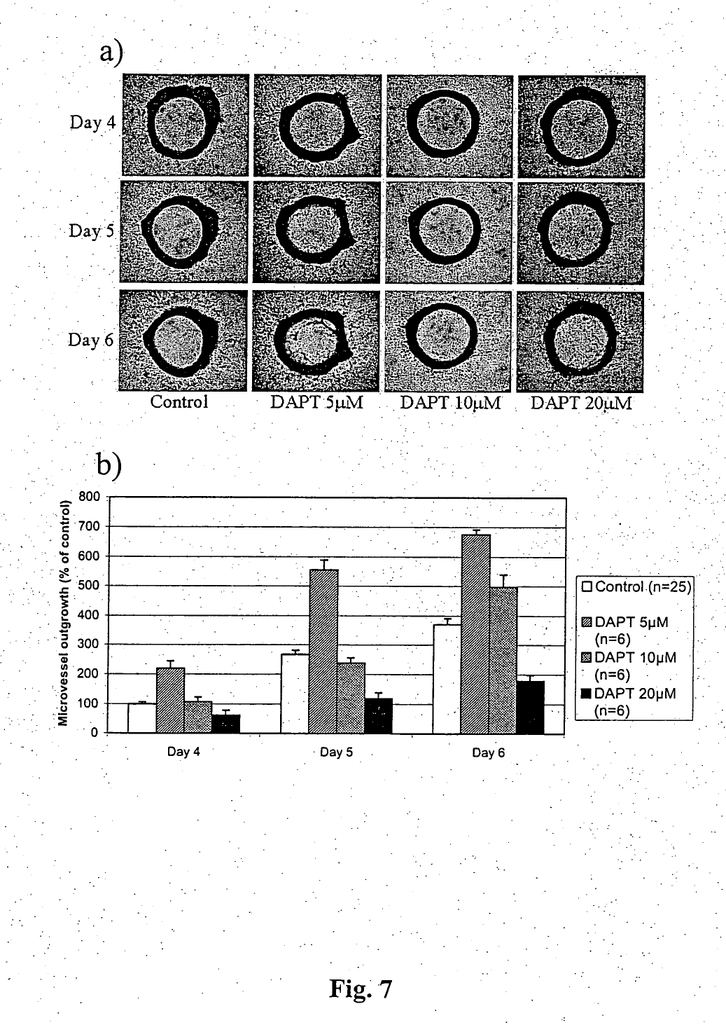

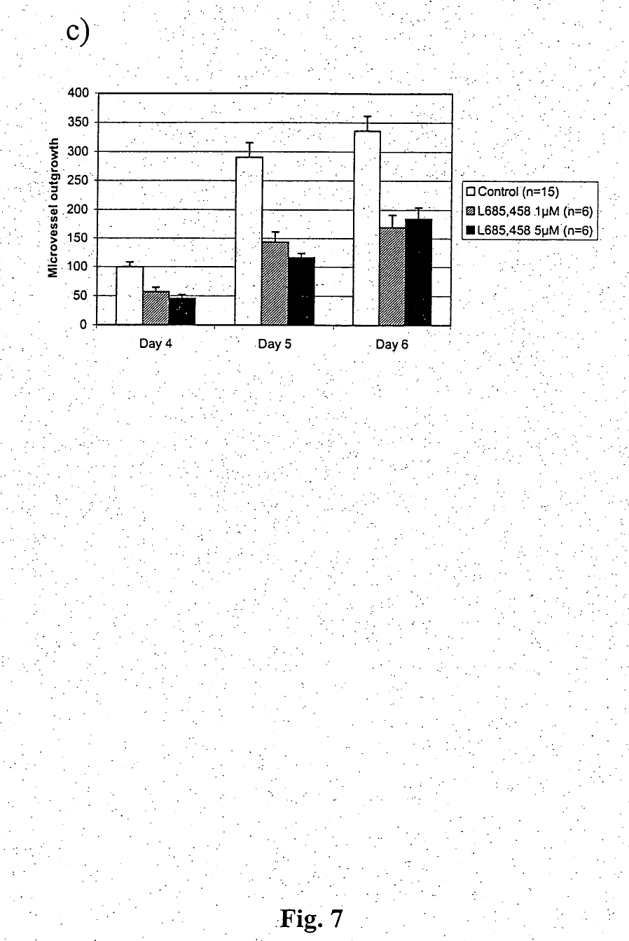

The γ-secretase inhibitor DAPT appeared to stimulate the sprouting of microvessels at 5 μM and to inhibit the sprouting of microvessels at 20 μM (FIG. 7). Additionally, the γ-secretase inhibitor L-685,458 inhibited the sprouting at 1 and 5 μM, further supporting the involvement of a γ-secretase-like activity during angiogenesis (FIG. 7).

### EXAMPLE 3

### Effect of the β-Secretase Inhibitor Z-VLL-CHO and the γ-secretase Inhibitor DAPT on the Growth of Tumor Xenografts in Nude Mice

An investigation was undertaken to determine the effects of the dipeptide protease γ-secretase inhibitor DAPT and the substrate analogue peptide β-secretase inhibitor Z-VLL-CHO, on the growth of human glioblastoma U-87 MG tumor cells, xenografted under the skin of nude mice.

### Materials and Methods

The human glioblastoma U-87 MG and human lung adenocarcinoma A-549 cell lines were obtained from American Tissue Culture Type Collection (Manassas, Va.) and were grown in DMEM containing 1×penicilline-streptomycine-fungizone and 10% fetal bovine serum at 37° C. in a humidified atmosphere of 5% CO₂. Tumor cells (6×106) in 100 μl of PBS were inoculated subcutaneously into both flanks of 8-10-week-old female nude mice (Harland). Tumor volume (in mm³) was determined using the formula (length×width²)/2, where length was the longest axis and width the measurement at right angles to the length (Clarke et al. 2000). When the tumor volumes reached approximately 150 mm³, animals were treated intraperitoneally everyday with 100 μl of 50% DMSO/H₂O (vehicle group), 5 mg/Kg of body weight of Z-VLL-CHO (β-secretase inhibitor), 5 mg/Kg of JLK-6 (γ-secretase inhibitor) or with 10 mg/Kg of body weight of DAPT. Data were expressed as mean tumor volume±S.E for each treatment group.

At the completion of the study, animals were humanely euthanized and tumors were harvested and fixed in paraformaldehyde 4% overnight at 4° C. After paraffin embedding in an automated tissue processing Sakura Tissue-Tek (E150) (Torrence, Calif.), samples were cut into 5 μm sections, deparafinized, and rehydrated through a graded series of alcohol. Sections were treated with 0.02 mg/ml Proteinase K (Gentra Systems, MN) for 15 minutes at 37° C. to allow for proper antigen retrieval, washed several times in PBS and incubated for 15 minutes in a 0.3% solution of hydrogen peroxide. Sections were blocked and then immunostained with a 1:40 dilution of a PECAM-1 antibody (BD-Pharmingen, CA) overnight at 4° C. in a humidification chamber. Vector ABC Kits (Vector Laboratories Inc, CA) where used following the manufacturer's instruction for the immunostaining. Quantification of tumor vascularization was performed using the stereological dissector method. Briefly, forty consecutive sections were taken from a randomly chosen starting point in each tumor. Five sections for each tumor were selected for stereology by taking one section every eight sections. A dissector counting frame was used with inclusion and exclusion lines throughout the reference area. Vessel count was performed at ×400 magnification with the use of an Olympus microscope connected to a digital video camera. Microvessels were counted in the dissector frame by an experimenter unaware of the different treatment conditions. For each tumor an average vessel count per area of dissector frame was determined. A vascular index was calculated by expressing the vessel count as a percentage of the vessel count in the vehicle treatment condition.

### Results

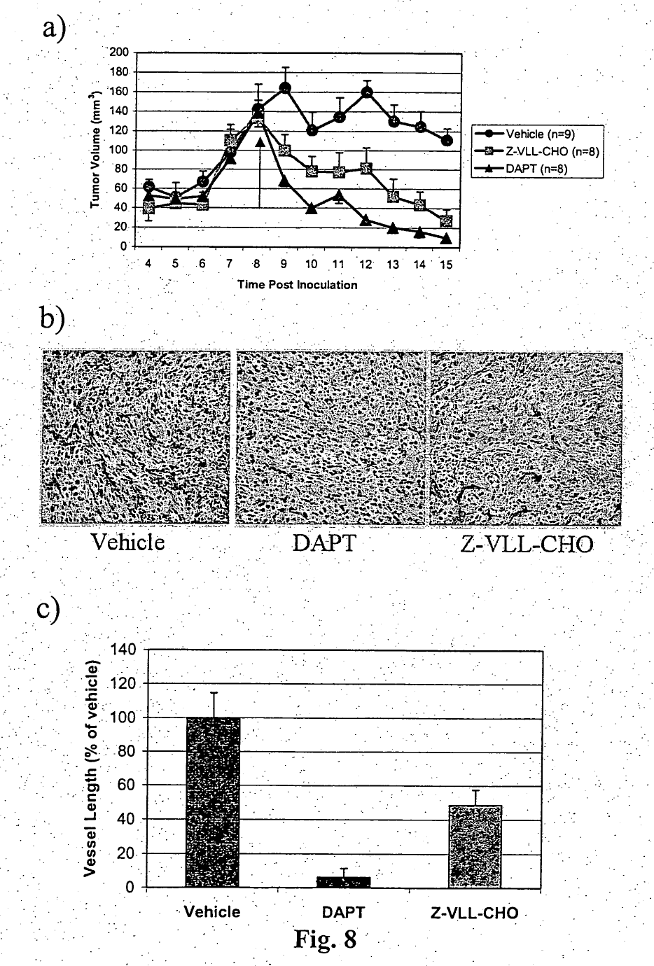

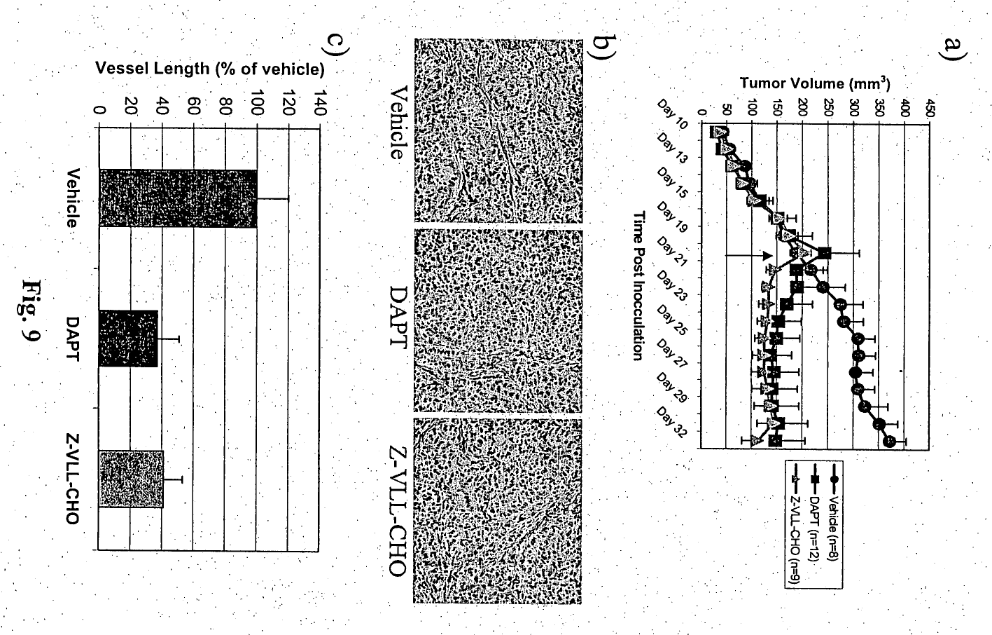

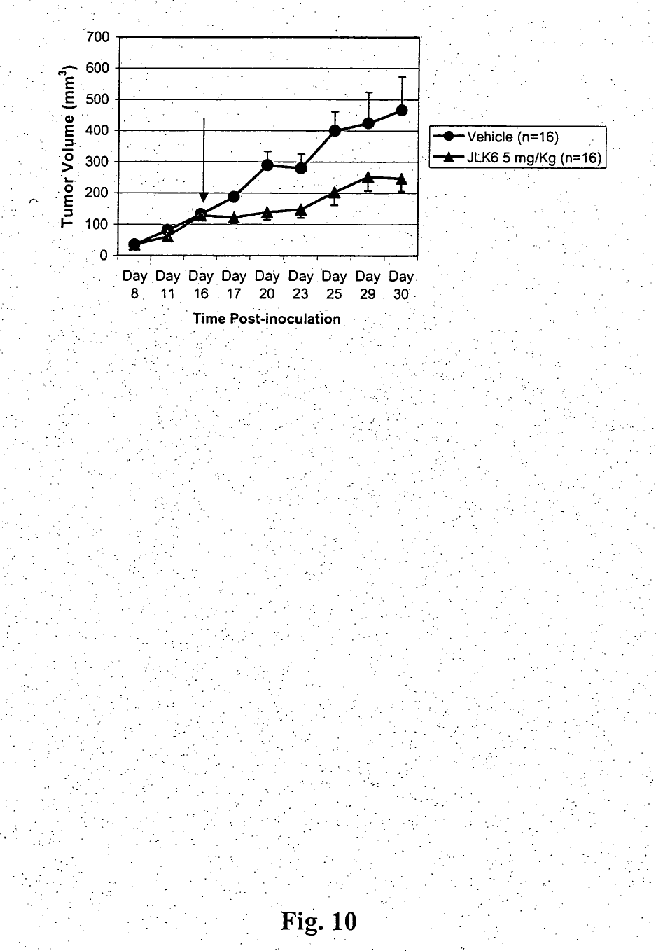

Both the β-secretase inhibitor, Z-VLL-CHO, and the γ-secretase inhibitor, DAPT, not only completely inhibited the growth of U-87 MG brain tumors (FIG. 8), but also reduced the volume of the tumors by more than 90% after one week of treatment. Additionally, both Z-VLL-CHO and DAPT dose dependently inhibited the proliferation of U-87 MG brain tumor cells (FIG. 9). Vascularization of the tumors was evaluated by PECAM-1 immunostaining. A decreased vascularization was observed in U-87 MG tumors treated with DAPT and Z-VLL-CHO compared with the vehicle treatment group, suggesting that both DAPT and Z-VLL-CHO were able to inhibit tumor angiogenesis in vivo. Z-VLL-CHO and DAPT dose dependently inhibited the proliferation of the tumor cells but did not display tumoricidal activity. The effect of DAPT and Z-VLL-CHO on the growth of the human lung adenocarcinoma cell line A-549 revealed that both compounds potently suppressed the growth of A-549 lung adenocarcinoma tumors in nude mice (FIG. 9). Additionally, the vascularization of A-549 tumors appeared to be decreased following DAPT or Z-VLL-CHO treatment, suggesting in vivo inhibition of angiogenesis by DAPT and Z-VLL-CHO. Finally, the γ-secretase inhibitor, JLK-6, inhibited the growth and vascularization of human lung adenocarcinoma tumors xenotransplanted into nude mice (FIG. 10).

It should be understood that the embodiments described herein are for illustrative purposes only and that various modifications or changes in light thereof will be suggested to persons skilled in the art and are to be included within the spirit and purview of this application.

The invention claimed is:

1. A method of treating tumors or proliferative disorders in an animal or human in need of such treatment, comprising administering to the animal or human therapeutically effective amounts in unit dosage form of a composition comprising a carrier and at least one secretase inhibitor.

2. The method of claim 1, wherein the secretase inhibitor specifically inhibits amyloid precursor protein secretases.

3. The method of claim 2, wherein the secretase inhibitor is a γ-secretase inhibitor.

4. The method of claim 3, wherein the γ-secretase inhibitor is an aspartyl protease transition-state γ-secretase inhibitor having the following backbone chemical structure: 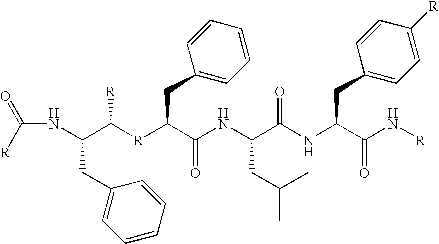
wherein R refers to analogue substitutions.

5. The method of claim 4, wherein the aspartyl protease transition-state γ-secretase inhibitor is L-685,458.

6. The method of claim 3, wherein the γ-secretase inhibitor is a dipeptide protease γ-secretase inhibitor having the following two backbone structures: 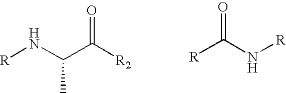
wherein R refers to analogue substitutions.

7. The method of claim 6, wherein the dipeptide protease γ-secretase inhibitor is selected from the group consisting of DAPT and DAPM.

8. The method of claim 3, wherein the γ-secretase inhibitor is an isocoumarin-based serine protease γ-secretase inhibitor having the following backbone structure: 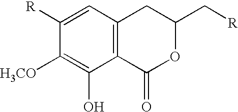
wherein R refers to analogue substitutions.

9. The method of claim 8, wherein the isocoumarin-based serine protease γ-secretase inhibitor is JLK-6.

10. The method of claim 2, wherein the secretase inhibitor is a β-secretase inhibitor having the following chemical structure: 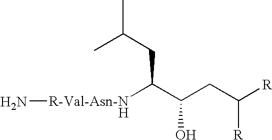
wherein R refers to analogue substitutions.

11. The method of claim 10, wherein the β-secretase inhibitor is a peptidomimetic tight binding transition-state analogue β-secretase inhibitor.

12. The method of claim 11, wherein the peptidomimetic tight binding transition-state analogue β-secretase inhibitor is OM99-2.

13. The method of claim 10, wherein the β-secretase inhibitor is a substrate analogue peptide β-secretase inhibitor.

14. The method of claim 13, wherein the substrate analogue peptide β-secretase inhibitor is selected from the group consisting of Z-VLL-CHO, GL189 and P10-P4′statV.

15. The method of claim 1, wherein the tumors are selected from the group consisting of glioblastomas, lung adenocarcinomas and malignant tumors of the breast, colon, kidney, bladder, head or neck.

16. The method of claim 1, wherein the proliferative disorders are hematopoietic disorders.

17. The method of claim 16, wherein the hematopoietic disorders are selected from the group consisting of leukemias, lymphomas and polycythemias.

18. The method of claim 1, wherein the proliferative disorders are ocular disorders.

19. The method of claim 18, wherein the ocular disorders are selected from the group consisting of diabetic retinopathy, macular degeneration, glaucoma and retinitis pigmentosa.

20. The method of claim 1, wherein the carrier is a pharmaceutically acceptable carrier or diluent.

21. The method of claim 1, wherein the route of administration of the composition to the animal or human is via parenteral, oral or intraperitoneal administration.

22. The method of claim 21, wherein the parenteral route of administration is selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, and nasal inhalation via insufflation or nebulization.

23. The method of claim 1, wherein the unit dosage is administered orally in the form of hard or soft shell gelatin capsules, tablets, troches, sachets, lozenges, elixirs, suspensions, syrups, wafers, powders, granules, solutions or emulsions.

24. The method of claim 22, wherein the nasal administration of the secretase inhibitor is selected from the group consisting of aerosols, atomizers and nebulizers.

25. A method of inhibiting angiogenesis associated with tumors, proliferative disorders or inflammatory disorders in an animal or human in need of such inhibition, comprising administering to the animal or human therapeutically effective amounts in unit dosage form of a composition comprising a carrier and at least one secretase inhibitor.

26. The method of claim 25, wherein the secretase inhibitor specifically inhibits amyloid precursor protein secretases.

27. The method of claim 26, wherein the secretase inhibitor is a γ-secretase inhibitor.

28. The method of claim 27, wherein the γ-secretase inhibitor is an aspartyl protease transition-state γ-secretase inhibitor having the following backbone structure: 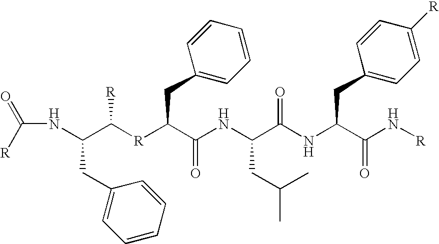
wherein R refers to analogue substitutions.

29. The method of claim 28, wherein the aspartyl protease transition-state γ-secretase inhibitor is L-685,458.

30. The method of claim 27, wherein the γ-secretase inhibitor is a dipeptide protease γ-secretase inhibitor having the following two backbone structures: 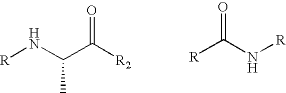
wherein R refers to analogue substitutions.

31. The method of claim 30, wherein the dipeptide protease γ-secretase inhibitor is selected from the group consisting of DAPT and DAPM.

32. The method of claim 27, wherein the γ-secretase inhibitor is an isocoumarin-based serine protease γ-secretase inhibitor having the following backbone chemical structure: 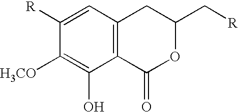
wherein R refers to analogue substitutions.

33. The method of claim 32, wherein the isocoumarin-based serine protease γ-secretase inhibitor is JLK-6.

34. The method of claim 26, wherein the secretase inhibitor is a β-secretase inhibitor having the following chemical structure: 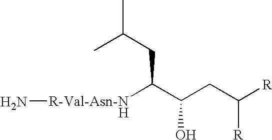
wherein R refers to analogue substitutions.

35. The method of claim 34, wherein the β-secretase inhibitor is a peptidomimetic tight binding transition-state analogue β-secretase inhibitor.

36. The method of claim 35, wherein the peptidomimetic tight binding transition-state analogue β-secretase inhibitor is OM99-2.

37. The method of claim 34, wherein the β-secretase inhibitor is a substrate analogue peptide β-secretase inhibitor.

38. The method of claim 37, wherein the substrate analogue peptide β-secretase inhibitor is selected from the group consisting of Z-VLL-CHO, GL189 and P10-P4′statV.

39. The method of claim 25, wherein the tumors are selected from the group consisting of glioblastomas, lung adenocarcinomas and malignant tumors of the breast, colon, kidney, bladder, head or neck.

40. The method of claim 25, wherein the proliferative disorders are hematopoietic disorders.

41. The method of claim 40, wherein the hematopoietic disorders are selected from the group consisting of leukemias, lymphomas and polycythemias.

42. The method of claim 25, wherein the proliferative disorders are ocular disorders.

43. The method of claim 42, wherein the ocular disorders are selected from the group consisting of diabetic retinopathy, macular degeneration, glaucoma and retinitis pigmentosa.

44. The method of claim 26, wherein the inflammatory disorders are selected from the group consisting of rheumatoid arthritis, osteoarthritis, pulmonary fibrosis, sarcoid granulomas, psoriasis and asthma.

45. The method of claim 25, wherein the carrier is a pharmaceutically acceptable carrier or diluent.

46. The method of claim 25, wherein the route of administration of the composition to the animal or human is via parenteral, oral or intraperitoneal administration.

47. The method of claim 46, wherein the parenteral route of administration is selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, and nasal inhalation via insufflation or nebulization.

48. The method of claim 25, wherein the unit dosage is administered orally in the form of hard or soft shell gelatin capsules, tablets, troches, sachets, lozenges, elixirs, suspensions, syrups, wafers, powders, granules, solutions or emulsions.

49. The method of claim 46, wherein the nasal administration of the secretase inhibitor is selected from the group consisting of aerosols, atomizers and nebulizers.
